(19) United States
(12) Patent Application Publication
(10) Pub. No.: US 2025/0387150 A1
(43) Pub. Date: Dec. 25, 2025

(54) INTRA-OSSEOUS PLATE SYSTEM AND METHOD

(71) Applicant: Treace Medical Concepts, Inc., Ponte Vedra, FL (US)

(72) Inventors: David L. Brumfield, Collierville, TN (US); Paul Dayton, Ankeny, IA (US); F. Barry Bays, Clayton, GA (US); Joe W. Ferguson, Ponte Vedra Beach, FL (US)

(21) Appl. No.: 19/309,156

(22) Filed: Aug. 25, 2025

Related U.S. Application Data
(63) Continuation of application No. 18/649,799, filed on Apr. 29, 2024, now Pat. No. 12,396,771.
Application No. 18/649,799 is a continuation of application No. 17/897,321, filed on Aug. 29, 2022, now Pat. No. 11,969,193.
Application No. 17/897,321 is a continuation of application No. 16/877,159, filed on May 18, 2020, now Pat. No. 11,426,219.
Application No. 16/877,159 is a continuation of application No. 15/148,774, filed on May 6, 2016, now Pat. No. 10,653,467.
(60) Provisional application No. 62/157,561, filed on May 6, 2015.

(51) Int. Cl. A61B 17/80; A61B 17/064; A61B 17/68; A61B 17/86; A61B 17/88

(52) U.S. Cl. CPC A61B 17/8061; A61B 17/686; A61B 17/8014; A61B 17/8095; A61B 17/8872; A61B 17/0642; A61B 17/86

(57) ABSTRACT

An intra-osseous support structure can be used to fixate opposed portions of bone. In some examples, the intra-osseous support structure is positioned in openings formed in adjacent portions of bones. Fasteners are inserted through the bone portions to secure the intra-osseous support structure in the bones. Depending on the application, one or more external bone plates may also be applied to the bone portions. The external bone plate may be in compression while the intra-osseous support structure is in tension under load in situ.

### RELATED APPLICATION

This application is a continuation of U.S. patent application Ser. No. 18/649,799, filed Apr. 29, 2024, which is a continuation of U.S. patent application Ser. No. 17/897,321, filed Aug. 29, 2022 and issued as U.S. Pat. No. 11,969,193, on Apr. 30, 2024, which is a continuation of U.S. patent application Ser. No. 16/877,159, filed May 18, 2020 and issued as U.S. Pat. No. 11,426,219, on Aug. 30, 2022, which is a continuation of U.S. patent application Ser. No. 15/148,774, filed May 6, 2016 and issued as U.S. Pat. No. 10,653,467, on May 19, 2020, which claims the benefit of U.S. Provisional Application Ser. No. 62/157,561, filed May 6, 2015. The entire contents of each of these applications are incorporated herein by reference.

### TECHNICAL FIELD

This disclosure relates generally to bone plate devices and methods for fixing bone using plate devices.

### BACKGROUND

Bones, such as the bones of a foot, may be anatomically misaligned. In certain circumstances, surgical intervention is required to correctly align the bones to reduce patient discomfort and improve patient quality of life. Surgical intervention may involve cutting one or more of the misaligned bones and then physically realigning the bones into an anatomically corrected position. A bone plate or multiple bone plates may be used to hold the bones in the anatomically corrected position, helping to prevent the bones from shifting back to their misaligned position.

### SUMMARY

In general, this disclosure is directed to bone fixation systems and techniques for fixating bones. In some examples, a bone plating system includes an intra-osseous support structure configured to be placed in an opening formed between adjacent bones. For example, during a tarsal-metatarsal fusion procedure in which a first metatarsal is realigned with respect to a second metatarsal, the intra-osseous support structure may be placed within the osseous tissue of the first metatarsal and the medial cuneiform, spanning the tarsal-metatarsal joint. An opening or groove may be formed in the end of the first metatarsal facing the medial cuneiform and also in the end of the medial cuneiform facing the first metatarsal, providing cavities in which opposed ends of the intra-osseous support structure are inserted. One or more fasteners can be used to secure the intra-osseous support structure to the bones in which the fastener is inserted. For instance, in the example of a tarsal-metatarsal fusion procedure, a fastener may be inserted into the medial cuneiform (e.g., from the dorsal toward the plantar side), securing the intra-osseous support structure to the medial cuneiform. A second fastener can be inserted into the first metatarsal (e.g., from the dorsal toward the plantar side), securing the intra-osseous support structure to the first metatarsal.

In some applications, a bone plate is also applied on exterior surfaces of the bone portions into which the intra-osseous support structure is inserted. For example, one or more flat or curved bone plates may be applied to exterior surfaces of bone portions containing the intra-osseous support structure. Depending on the configuration, the exterior bone plate(s) may be in compression while the intra-osseous support structure is in tensions under load, providing a balanced fixation system to effectively fixation opposed portions of bone.

In one example, a bone plating system is described that includes a fastener having a length and an intra-osseous support structure. The example specifies that the intra-osseous support structure is configured to be placed in an opening formed in a first bone portion and a second bone portion and has an aperture to receive the fastener.

In another example, a method of plating a bone is described. The method includes forming an opening in a first bone portion and a second bone portion and placing an intra-osseous support structure in the opening. The method further includes inserting a first fastener through the first bone portion and into the intra-osseous support structure and inserting a second fastener through the second bone portion and into the intra-osseous support structure.

### BRIEF DESCRIPTION OF THE DRAWINGS

FIG. 1 is a perspective view of a bone plating system in accordance with an embodiment of the invention.

FIG. 2 is a perspective view of a bone plating system in accordance with an embodiment of the invention.

FIG. 3 is a perspective view of a bone plating system in accordance with an embodiment of the invention.

FIG. 4 is a perspective view of a bone plating system in accordance with an embodiment of the invention, with bone portions depicted as transparent.

FIG. 5 is a top view of a bone plating system in accordance with an embodiment of the invention, with bone portions depicted as transparent.

FIG. 6 is a perspective view of a bone plating system in accordance with an embodiment of the invention, with bone portions depicted as transparent.

FIG. 7 is a perspective view of a bone plating system in accordance with an embodiment of the invention, with bone portions depicted as transparent.

FIG. 8 is a perspective view of a bone plating system in accordance with an embodiment of the invention, with bone portions depicted as transparent.

FIG. 9 is a side view of a bone plating system in accordance with an embodiment of the invention, with bone portions depicted as transparent.

FIG. 10 is an end view of a bone plating system in accordance with an embodiment of the invention, with bone portions depicted as transparent.

FIG. 11 is a side view of a bone plating system in accordance with an embodiment of the invention, with bone portions depicted as transparent.

FIG. 12 is an end view of a bone plating system in accordance with an embodiment of the invention, with bone portions depicted as transparent.

FIG. 13 is a top plan view of an intra-osseous support structure in accordance with an embodiment of the invention.

FIG. 14 is a top plan view of an intra-osseous support structure in accordance with an embodiment of the invention.

FIG. 15 is a perspective view of a bone plating system in accordance with an embodiment of the invention, with bone portions depicted as transparent.

FIG. 16 is a perspective view of a bone plating system in accordance with an embodiment of the invention, with bone portions depicted as transparent.

### DETAILED DESCRIPTION

The following detailed description is exemplary in nature and is not intended to limit the scope, applicability, or configuration of the invention in any way. Rather, the following description provides some practical illustrations for implementing exemplary embodiments of the present invention. Examples of constructions, materials, dimensions, and manufacturing processes are provided for selected elements, and all other elements employ that which is known to those of ordinary skill in the field of the invention. Those skilled in the art will recognize that many of the noted examples have a variety of suitable alternatives.

Embodiments of the invention include a bone plating system. Embodiments of the system can be useful for providing structural support to bones subject to a surgical procedure, such as a bone alignment, osteotomy, fracture repair, and/or fusion procedure. Such a procedure may be performed, for example, on bones (e.g., adjacent bones separated by a joint or different portions of a single bone separated by a fracture) in the foot or hand. In one example, the procedure can be performed to correct an alignment between a metatarsal (e.g., a first metatarsal) and a cuneiform (e.g., a first cuneiform), such as a bunion correction. An example of such a procedure is a lapidus procedure. In another example, the procedure can be performed by modifying an alignment of a metatarsal (e.g., a first metatarsal). An example of such a procedure is a basilar metatarsal osteotomy procedure.

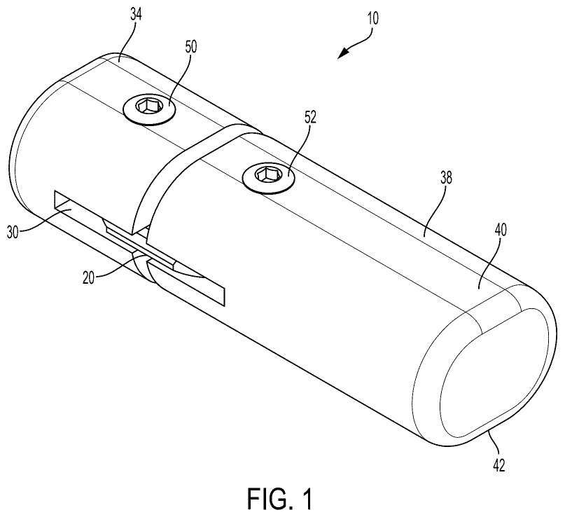

As shown in FIG. 1, embodiments of the bone plating system 10 include an intra-osseous support structure 20. As shown, the intra-osseous support structure can be adapted to be positioned intra-osseously. In such embodiments, the intra-osseous support structure is positioned within a thickness of a bone, such that both of its major surfaces face bone (e.g., cancellous bone). In the embodiment shown, the intra-osseous support structure 20 is adapted to be positioned within an opening 30 of a first bone portion 34 and a second bone portion 38, the opening leading to a cavity or void within the respective bone portions. As shown, in a bone portion having a dorsal surface 40 and a plantar surface 42, the opening can be formed closer to the plantar surface than the dorsal surface (e.g., between about one-half and two-thirds through the thickness of the bone). In certain embodiments, the opening crosses a centerline of one or both bone portions. In such embodiments, the bone defining the opening, and the intra-osseous support structure placed therein, will be in tension under load in situ. Such an intra-osseous support structure can be useful for providing structural support to bones subject to a surgical procedure.

The intra-osseous support structure 20 can include any useful form. In some embodiments, the intra-osseous support structure has a first major surface, a second major surface, and a perimeter edge extending between the first major surface and the second major surface. In the embodiment shown in FIG. 1, the intra-osseous support structure is generally planar as are its first and second major surfaces. The major surfaces can be devoid of any protrusions. In certain embodiments, one or both of the major surfaces can include a surface treatment such as a texture. In some embodiments (not shown), the intra-osseous support structure can include a portion generally perpendicular to a first major surface. For example, the intra-osseous support structure can include a portion that extends from a side (e.g., a medial side) and bends or curves in an upward (e.g., dorsal) or a downward (e.g. plantar) direction around and/or in apposition to a cortical surface of a bone.

In the embodiment shown in FIG. 1, the intra-osseous support structure 20 is connected to a bone portion by at least one fastener 50. In some embodiments, the intra-osseous support structure has a first portion for placement in the first bone portion 34 and a second portion for placement in the second bone portion 38, and at least one aperture (not shown in FIG. 1) to receive a respective fastener can be provided on each portion. In situ, the fastener can extend through a bone surface (e.g., a dorsal surface) and a portion of the thickness of a bone to the intra-osseously positioned support structure and be received into an aperture thereof.

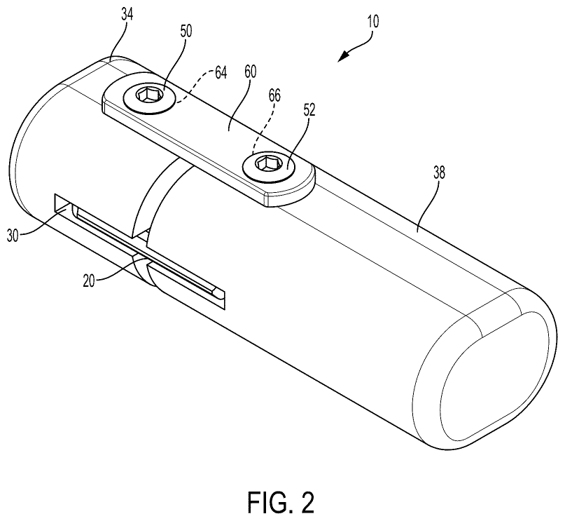

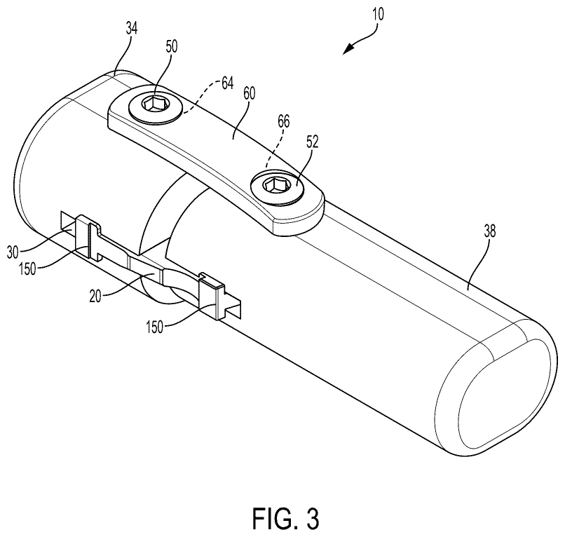

As shown in FIGS. 2 and 3, some embodiments of the bone plating system 10 can include a bone plate 60. In the embodiment shown, the plate is adapted to be positioned on an outer surface of the first bone portion 34 and an outer surface of the second bone portion 38. As shown, in a bone portion having a dorsal surface and a plantar surface, the dorsal surface, and the plate placed thereon, will be in compression under load in situ. Accordingly, some embodiments of the plating system include a plate in compression and an intra-osseous support structure in tension under load in situ. Such a plating system can be useful for providing structural support to bones subject to a surgical procedure.

The bone plate 60 can include any suitable form. In some embodiments, the bone plate has a bone facing surface and a surface opposite the bone facing surface. In certain embodiments, such as the embodiment shown in FIG. 2, the bone plate 60 includes a generally planar member having generally planar surfaces. In other embodiments, such as the embodiment shown in FIG. 3, the bone plate 60 includes a curved shape (about and/or along its longitudinal axis). For example, the surface facing the bone can be concave and the opposite surface can be convex. In certain embodiments, the surface of the bone plate facing the bone may also have at least one protrusion to engage with the surface of the bone.

In embodiments of the plating system having a plate 60, the plate and intra-osseous support structure 20 can be connected to the bone and each other by the at least one fastener 50. In such embodiments, the bone plate 60 can have at least one aperture 64, 66 to receive respective fasteners 50, 52. In the embodiment shown, the bone plate has a first portion for placement on the first bone portion 34 and a second portion for placement on the second bone portion 38. At least one aperture 64, 66 for receiving a respective fastener 50, 52 can be provided on each portion. Further, the intra-osseous support structure 20 can have at least one aperture (not shown in FIGS. 2 and 3) aligned to receive the respective fastener. In situ, the fastener can extend through the bone plate, a surface of the bone, and a portion of the thickness of a bone to the intra-osseously positioned support structure and be received into the aperture thereof. After final placement, in some embodiments, the bone plate and intra-osseous support structure will be generally parallel to each other.

In some embodiments, the aperture in the intra-osseous support structure can include an attachment mechanism configured to engage a fastener. The fastener and attachment mechanism can include any structure suitable for engagement. In some embodiments, the fastener includes a screw, and the attachment mechanism includes a threaded aperture to receive and engage the screw. The attachment mechanism can include guides to facilitate alignment with the fasteners. In some embodiments, the fastener has a length that is less than the thickness of the bone. In certain embodiments, the fastener will have a length between about one-half of the thickness of the bone and the entire thickness of the bone. For example, the fastener can have a length of about two-thirds the thickness of the bone. In some embodiments, the fastener can extend through the aperture of the intra-osseous support structure (optionally engaging an attachment member thereof) and engage bone on one or both sides of the intra-osseous support structure.

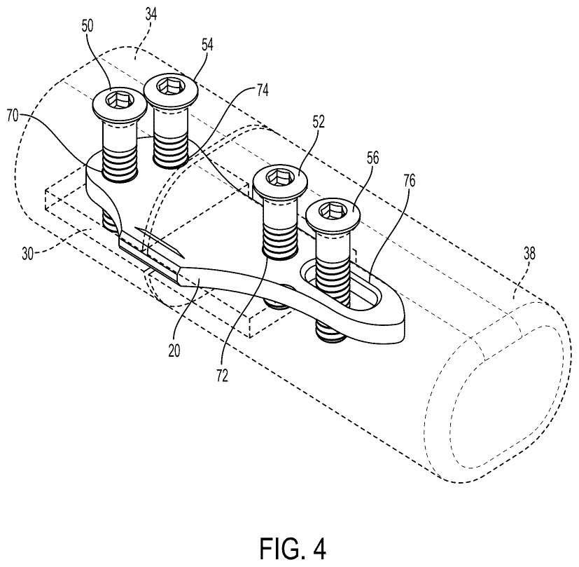

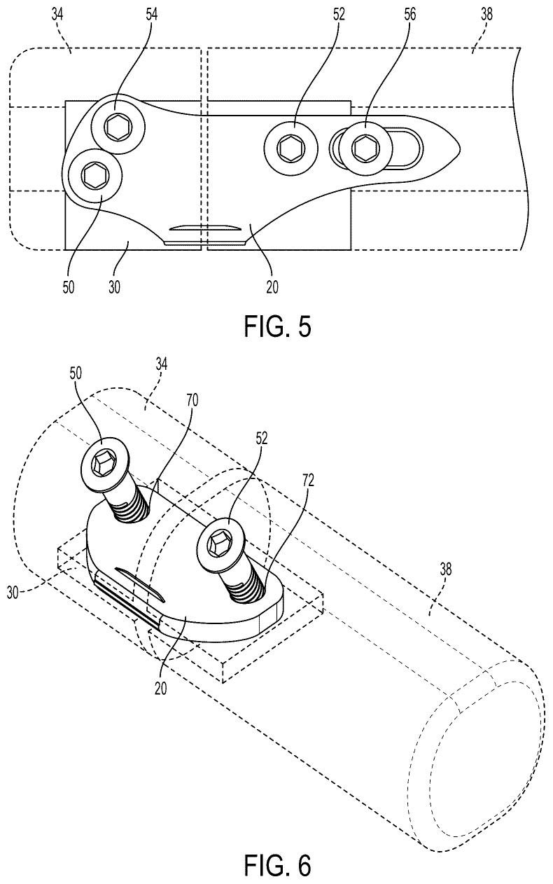

Any number of fasteners and respective intra-osseous support structure apertures can be provided. In the embodiment shown in FIGS. 1-3, the intra-osseous support structure 20 includes two apertures to receive two respective fasteners 50, 52. In the embodiment shown in FIGS. 4 and 5, the intra-osseous support structure 20 includes four apertures 70, 72, 74, 76 to receive four respective fasteners 50, 52, 54, 56. In other embodiments, the intra-osseous support structure includes three apertures to receive three respective fasteners. In an embodiment of an intra-osseous support structure having a portion generally perpendicular to a first major surface, such a portion may include one or more apertures for receiving a fastener.

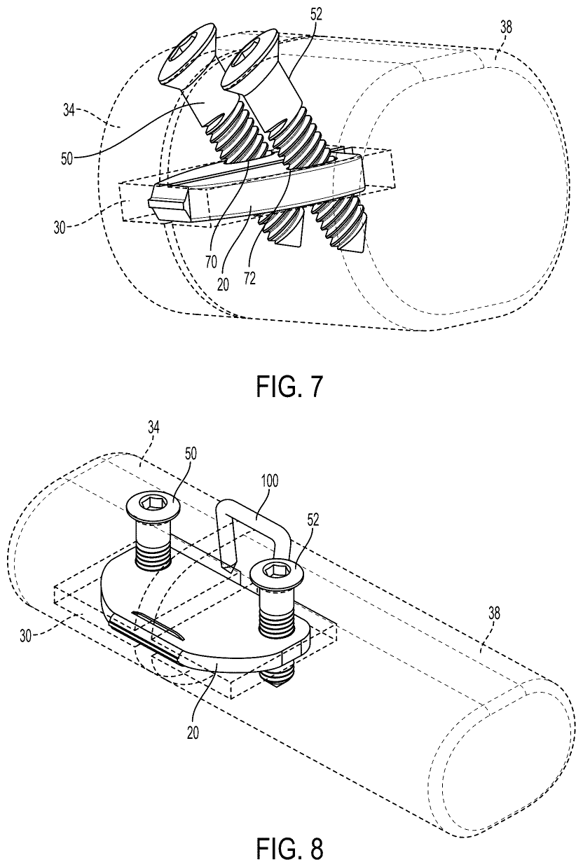

The fasteners and respective apertures can be provided in any orientation. In some embodiments, such as the embodiments shown in FIG. 4, the apertures 70, 72, 74, 76 have a longitudinal axis perpendicular to a first major surface of the intra-osseous support structure 20. In other embodiments, such as the embodiment shown in FIGS. 6 and 7, the apertures 70, 72 have a longitudinal axis that intersects a first major surface of the intra-osseous support structure 20 at a skewed angle (e.g., an angle ranging from about 20 degrees to about 40 degrees from perpendicular). Further, in some embodiments, fasteners 50, 52 can extend from the bone surface and through the bone generally parallel to each other. In other embodiments, first and second fasteners can extend from the bone surface and through the bone at a skewed angle relative to each other. In embodiments of the plating system that include a plate, apertures 64, 66 can be configured such that first and second fasteners 50, 52 can extend from the bone plate 60 generally parallel to each other or at a skewed angle relative to each other.

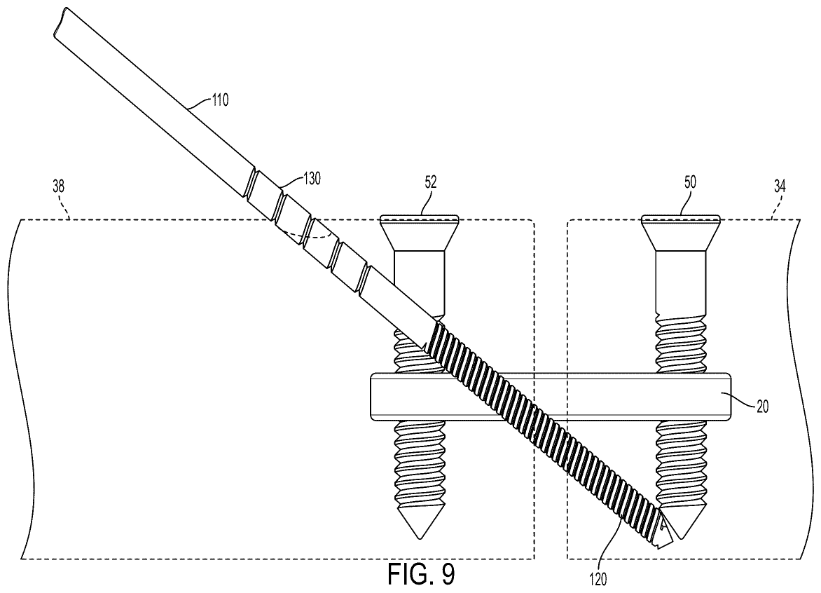

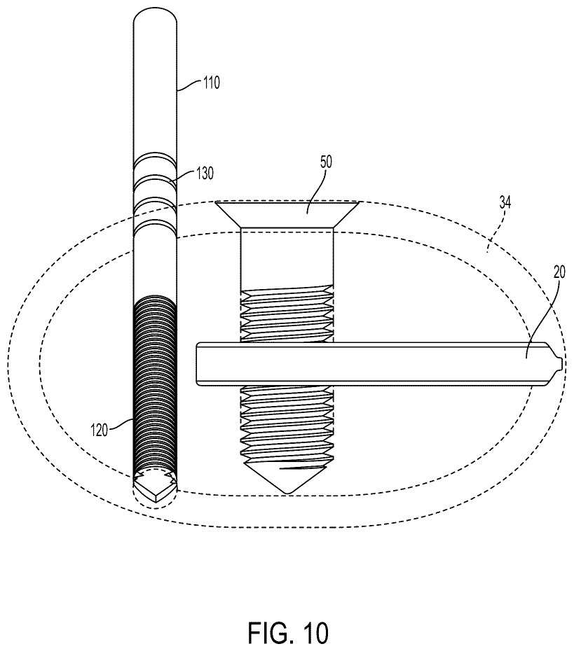

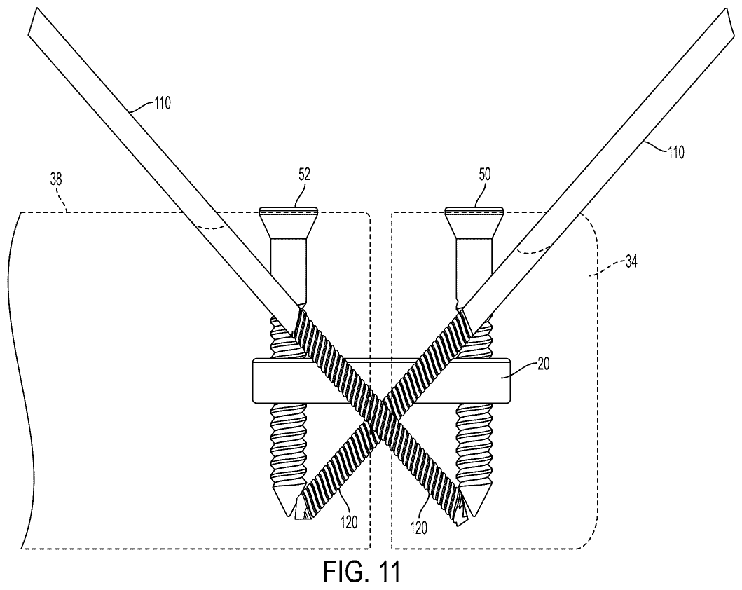

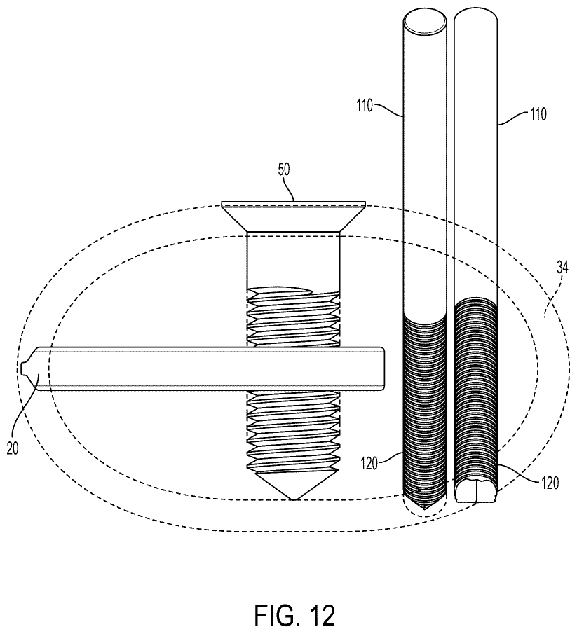

As shown in FIGS. 8-12, some embodiments of the plating system can include an additional support that does not engage the intra-osseous support structure. Such an additional support can be useful for providing rotational stability to the plated bone portions. In the embodiment shown in FIG. 8, the additional support includes a staple 100 having an end in each bone portion 34, 38. In the example shown in FIGS. 9-10, the additional support includes a pin 110 extending across the bone portions 34, 38 at an angle (e.g., about 45 degrees). In the embodiment shown, the pin 110 includes threads 120 on its distal portion to engage bone. It also includes notches 130 on its proximal portion. The pin can be broken at a desired notch after installation. In the embodiment shown in FIGS. 11 and 12, two pins 110 are provided. As shown, the pins are inserted such that they extend across the bone portions 34, 38 in a crossing pattern, each at an angle (e.g., about 45 degrees).

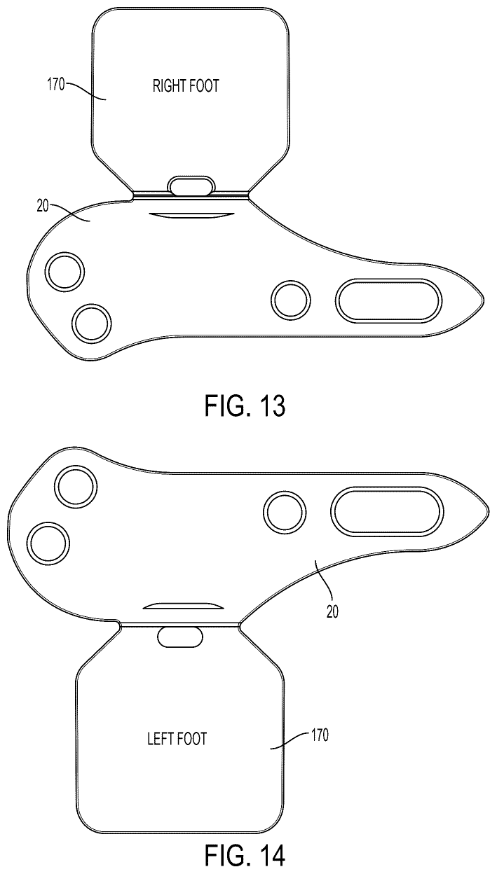

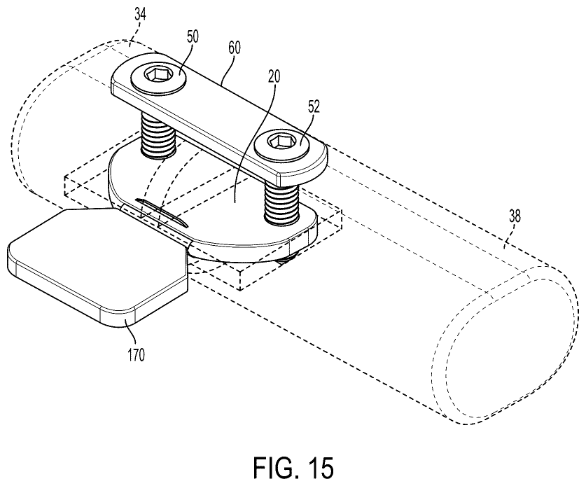

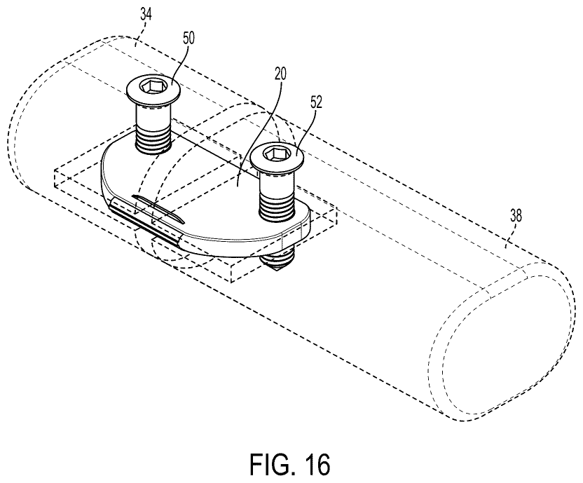

The plating system can also include features useful for placing the intra-osseous support structure. As shown in FIG. 3, the intra-osseous support structure 20 can include a stop 150 on the perimeter edge that extends past the first major surface or the second major surface. In some embodiments, the stop is on a medial side of the intra-osseous support structure. In other embodiments, as shown in FIG. 13-15, a tab 170 extending beyond the perimeter edge (e.g., on a medial side) can be provided. The tab can be useful for placing the intra-osseous support structure within the bone, and, in some embodiments, can be provided with break seam such that it can be easily removed from the intra-osseous support structure after placement. FIG. 16 shows in intra-osseous support structure 20 with the tab removed.

The plating system can be used to join any bone portions. In one example, the first bone portion and the second bone portion are portions of a single bone separated by a fracture. As a further example, the first bone portion and the second bone portion are portions of a single bone separated by an osteotomy. As another example, the first bone portion and the second bone portion can be portions of two different bones separated by a joint, such as a cuneiform (e.g., medial cuneiform) and a metatarsal (e.g., first metatarsal). In the two-bone example, the intra-osseous support structure can be placed intra-osseously in the cuneiform and the metatarsal in an opening that spans the joint therebetween (e.g., tarsal-metatarsal joint). In such an embodiment, fasteners having a length less than the thickness of the cuneiform and metatarsal, respectively, can be used to connect the intra-osseous support structure to the bones. In embodiments of the plating system having a bone plate, the bone facing surface of the bone plate can be placed facing a dorsal surface of the cuneiform and a dorsal surface of the metatarsal, spanning a joint therebetween, and the fasteners can extend through apertures defined by the plate.

Embodiments of the invention also include methods of plating a bone, such as with the embodiments of bone plating systems described herein. Note the order of steps as described is only exemplary unless otherwise indicated. In some embodiments, after preparing the surgical area, the method can include the step of forming an opening in a first bone portion and a second bone portion. The opening can be formed from a side of the bone. The opening can be formed generally parallel with a longitudinal axis of the bone, or may be formed at an angle with respect to such longitudinal axis such that it crosses the longitudinal axis. The opening can be formed, e.g., by a saw, drill, mill, box chisel, router, or the like.

The method can also include the steps of placing an intra-osseous support structure in the opening and aligning it in a desired position. In some embodiments, the intra-osseous support structure can be placed generally parallel to a longitudinal axis of the bone (e.g., toward a tension side of the longitudinal axis. In other embodiments, the intra-osseous support structure can be placed at a skewed angle relative to the longitudinal axis of the bone, such that it crosses the longitudinal axis of the bone. In such embodiments, at least a portion of the intra-osseous support structure will reside on a tension side of the longitudinal axis and another portion will reside on a compression side of the longitudinal axis. The method can also include the steps of inserting a first fastener through a first bone portion and engaging the first fastener with the intra-osseous support structure, and inserting a second fastener through a second bone portion and engaging the second fastener with the intra-osseous support structure to secure the plating system to the bone. In some embodiments, the step of placing the intra-osseous support structure in the opening includes placing a stop in apposition to the first bone portion or the second bone portion. In embodiments of intra-osseous support structures having tabs, the method can also include removing the tab after placement of the support structure. Embodiments of the method can also include attaching an additional support structure to the first bone portion and the second bone portion.

In some embodiments, the method can also include the step of forming a first hole in the first bone portion from the first surface and toward an opposite surface and forming a second hole in the second bone portion from the second surface and toward an opposite surface. The first and second holes and the opening can intersect. The first and second holes can be formed, for example, with hand-driven or powered drills. In such embodiments, the fasteners can be inserted through the holes to engage an intra-osseous support structure placed within the opening.

Embodiments of the method also include placing a bone plate having a first portion in apposition to a first surface of a first bone portion and a second portion in apposition to a second surface of a second bone portion, the bone plate having a first aperture in the first portion and a second aperture in the second portion. The bone plate can be initially held in position by pins and/or protrusions. The fasteners can be inserted through apertures defined by the plate.

Thus, embodiments of the invention are disclosed. Although the present invention has been described with reference to certain disclosed embodiments, the disclosed embodiments are presented for purposes of illustration and not limitation, and other embodiments of the invention are possible. One skilled in the art will appreciate that various changes, adaptations, and modifications may be made without departing from the spirit of the invention.

1. A method comprising:
performing an osteotomy on a metatarsal to separate the metatarsal into a first metatarsal portion and a second metatarsal portion;
adjusting an alignment of the first metatarsal portion relative to the second metatarsal portion;
placing a first portion of an intra-osseous support structure in the first metatarsal portion; and
connecting a second portion of the intra-osseous support structure to the second metatarsal portion using at least one fastener.

2. The method of claim 1, wherein connecting the second portion of the intra-osseous support structure to the second metatarsal portion comprises inserting a screw through an outer surface of the second metatarsal portion.

3. The method of claim 2, further comprising forming a hole in the second metatarsal portion from the outer surface toward an opposite surface, wherein inserting the screw through the outer surface of the second metatarsal portion comprises inserting the screw into the hole.

4. The method of claim 2, wherein the second portion of the intra-osseous support structure comprises a threaded aperture, and inserting the screw through the outer surface of the second metatarsal portion comprises inserting the screw into the threaded aperture.

5. The method of claim 1, wherein connecting the second portion of the intra-osseous support structure to the second metatarsal portion using at least one fastener comprises inserting the at least one fastener through a thickness of the second metatarsal portion less than an entire thickness of the second metatarsal portion.

6. The method of claim 5, wherein the thickness of the second metatarsal portion is within a range from one-half to two-thirds of the entire thickness of the second metatarsal portion.

7. The method of claim 1, further comprising inserting a fastener into the first portion of the intra-osseous support structure.

8. The method of claim 7, wherein inserting the fastener into the first portion of the intra-osseous support structure comprises inserting the fastener through an outer surface of the first metatarsal portion.

9. The method of claim 8, wherein inserting the fastener through the outer surface of the first metatarsal portion comprises engaging the fastener with the intra-osseous support structure in the first metatarsal portion.

10. The method of claim 7, wherein inserting the fastener into the first portion of the intra-osseous support structure comprises inserting the fastener through a thickness of the first metatarsal portion less than an entire thickness of the first metatarsal portion.

11. The method of claim 7, wherein the fastener comprises a first fastener, and further comprising inserting a second fastener into the first portion of the intra-osseous support structure.

12. The method of claim 1, further comprising placing the second portion of the intra-osseous support structure in the second metatarsal portion.

13. The method of claim 12, further comprising forming an opening in the second metatarsal portion, wherein placing the second portion of the intra-osseous support structure in the second metatarsal portion comprises placing the second portion of the intra-osseous support structure in the opening in the second metatarsal portion.

14. The method of claim 1, wherein placing the first portion of the intra-osseous support structure in the first metatarsal portion and connecting the second portion of the intra-osseous support structure to the second metatarsal portion comprises fusing the first metatarsal portion to the second metatarsal portion.

15. The method of claim 1, further comprising forming an opening in the first metatarsal portion, wherein placing the first portion of the intra-osseous support structure in the first metatarsal portion comprises placing the first portion of the intra-osseous support structure in the opening in the first metatarsal portion.

16. The method of claim 1, wherein the at least one fastener comprises two fasteners, and connecting the second portion of the intra-osseous support structure to the second metatarsal portion using at least one fastener comprises connecting the second portion of the intra-osseous support structure to the second metatarsal portion using two fasteners.

17. The method of claim 1, wherein the metatarsal is a first metatarsal.

18. A method comprising:
performing an osteotomy on a metatarsal to separate the metatarsal into a first metatarsal portion and a second metatarsal portion;
adjusting an alignment of the first metatarsal portion relative to the second metatarsal portion; and
fixating the first metatarsal portion relative to the second metatarsal portion for fusion using a structure that includes a first portion configured to be positioned intra-osseously and a second portion that includes at least one aperture configured to receive a screw, wherein fixating the first metatarsal portion relative to the second metatarsal portion comprises inserting the first portion of the structure within the first metatarsal portion and screwing at least one screw into the at least one aperture of the second portion of the structure and into the second metatarsal portion.

19. The method of claim 18, wherein inserting the first portion of the structure within the first metatarsal portion comprises inserting the first portion of the structure within cancellous bone of the first metatarsal portion.

20. The method of claim 18, wherein:
the metatarsal is a first metatarsal;
the at least one aperture comprises two apertures;
the at least one screw comprises two screws; and
screwing at least one screw into the at least one aperture of the second portion of the structure and into the second metatarsal portion comprises screwing two screws into the two apertures of the second portion of the structure and into the second metatarsal portion.
